(19) United States
(12) Patent Application Publication
(10) Pub. No.: US 2003/0068282 A1
(43) Pub. Date: Apr. 10, 2003

(54) Composition

(75) Inventors: Alison Katharine Green, Merseyside (GB); Peter John Hall, Merseyside (GB); David Thomas Littlewood, Merseyside (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/225,855

(22) Filed: Aug. 22, 2002

(30) Foreign Application Priority Data
Aug. 6, 2002 (EP) 02255497.6
Aug. 24, 2001 (EP) 01307269.9
Dec. 11, 2001 (EP) 01310338.7

(51) Int. Cl. A61K 7/16

(52) U.S. Cl. 424/49

(57) ABSTRACT

Topical composition comprising

(a) an alkyl hydroxybenzoate represented by formula 1

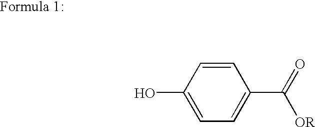

wherein R represents an alkyl group comprising at least five carbon atoms, and

(b) from 1.3 to 1.7% by weight of the composition a surfactant.

The present invention relates to a composition comprising an alkyl hydroxybenzoate and a surfactant.

### INTRODUCTION

Alkyl hydroxybenzoates (parabens) are known in the art where the alkyl group is methyl. For example, methyl hydroxybenzoate is mentioned, albeit fleetingly, for use in medicinal and oral care preparations as a preservative (WO 00/09507 and WO 00/69401).

In addition, U.S. Pat. No. 5,094,841 (Fine) discloses the use of heptyl paraben as a preservative in an oral care formulation. However, it also states that the preferred preservatives are methyl and propyl paraben and only ever states that they may be included in small amounts (0.1%) to provide a preservative effect.

EP-A2-0 161 898 (Unilever) discloses the use of parabens in oral care, in particular, methyl, ethyl, propyl, isopropyl, butyl, isobutyl, hexyl, heptyl and benzyl parabens. Aslo disclosed are levels of sodium lauryl sulphate at from 1 to 3% by weight of the composition.

We have found that there exists a surprising optimum in the levels of alkyl hydroxybenzoates when used in oral care compositions with surfactants. This is particularly the case where the skilled person in the art would expect the alkyl hydroxybenzoate to have a foam dampening effect, for example where the alkyl chain length comprises more than five carbon atoms.

### STATEMENT OF INVENTION

Accordingly, the invention provides oral composition comprising

(a) an alkyl hydroxybenzoate represented by formula 1

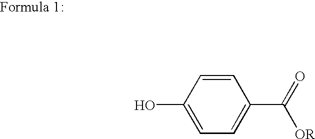

wherein R represents an alkyl group comprising at least five carbon atoms, and

(b) from 1.3 to 1.7% by weight of the composition a surfactant.

### DESCRIPTION OF INVENTION

The alkyl group of the compound according to formula 1 is an alkyl comprising more than five carbon atoms. Preferably, the alkyl group comprises no more than 30 carbon atoms. More preferably the alkyl group comprises from 6 to 15 carbon atoms, especially from 6 to 10 and especially preferably, 7 or 8.

Further, the alkyl group may be branched or straight chain and/or substituted or unsubstituted.

Preferred alkyl groups include octyl, heptyl and 2-ethylhexyl, more preferably, octyl or 2-ethylhexyl. Such compounds may be made by simple esterification of 4-hydroxybenzoic acid with the respective alcohol. Such a process is a simple step for the person skilled in the art to carry out.

The compound according to formula 1 is preferably present in an amount such that an antibacterial effect can be provided. In practice this ranges from 0.15 to 20% by weight of the composition according to the invention. Preferably, in an amount ranging from 0.8 to 5% by weight and even more suitably from 1.15 to 1.5% by weight. A level of 1.15% by weight of the composition up to 1.5% by weight in combination with a level of surfactant from 1.3 to 1.7% by weight provides an optimum antibacterial effect while still providing an acceptable level of foaming. This is the case even when the alkyl chain comprises six carbon atoms but is even more surprising where the alkyl chain comprises from seven to eleven carbon atoms. The most effective combination is where the alkyl chain of the alkyl para hydroxybenzoate comprises eight carbon atoms in a straight chain. This provides the best antimicrobial effect without inhibiting the foaming provided by from 1.3 to 1.7% by weight surfactant.

The surfactant in the oral composition is capable of foaming the composition during use. Preferred surfactants include the alkali-metal alkyl sulphates, especially sodium lauryl sulphate. The surfactant is present in the oral composition at from 1.3 to 1.7% by weight of the composition, preferably from 1.4 to 1.6% by weight.

It is to be understood that the oral composition according to the invention is capable of being used to clean the oral cavity, whether as part of a quotidian regime or as part of a one-off treatment. Typically oral care compositions comprise orally acceptable carriers. Further, oral compositions usually comprise oral care benefit agents selected from the group consisting of anti-caries agents, anti-tartar agents, flavours, whitening agents, abrasives, bleaches and anti-malodour agents.

The oral composition according to the invention may also comprise further ingredients which are common in the art, such as:

antimicrobial agents, e.g. Triclosan, chlorhexidine, copper-, zinc- and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition.

humectants such as glycerol, sorbitol, propyleneglycol, xylitol, lactitol etc.;

binders and thickeners such as sodium carboxymethyl-cellulose, xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

Embodiments according to the invention shall now be discussed with reference to the following non-limiting examples.

### EXAMPLE

Standard toothpastes comprising SLS and n-octyl paraben were tested in a salivary sediment model similar to that described by R. L. Wijeijweera and I. Kleinberg in Archs. Oral Biol., Vol. 34, No. 1, 1989, pages 43-53, using ex-vivo samples and measuring the amount of n-octyl paraben delivered to the salivary sediment.

Two repetitions were carried out for each SLS level in a paste comprising 1.1% n-octyl paraben. The values represnet the amount (ppm per ml sediment suspension) of paraben delivered.

TABLE 1

| % SLS in base | | Rep 1 | Rep 2 | Mean |
|---|---|---|---|---|
| | 1.4 | 60.080 | 61.657 | 60.869 |
| | 1.6 | 59.534 | 63.775 | 61.654 |
| | 1.8 | 48.790 | 48.352 | 48.571 |

As can be clearly seen the level of paraben delivered is greater at SLS levels ranging at 1.4 and 1.6% than for 1.8%. This is an unexpected effect.

1. Oral composition comprising
(a) an alkyl hydroxybenzoate represented by formula 1 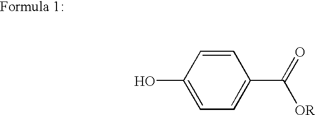
wherein R represents an alkyl group comprising at least five carbon atoms, and
(b) from 1.3 to 1.7% by weight of the composition a surfactant.

2. Composition according to claim 1, wherein R represents an alkyl group comprising from six to fifteen carbon atoms.

3. Composition according to claim 1, wherein R represents an alkyl group comprising from seven to ten carbon atoms.

4. Composition according to claim 1, wherein R represents a group selected from the group consisting of octyl and heptyl.

5. Composition according to claim 1, wherein R represents a branched alkyl group.

6. Composition according to claim 1, wherein R is a straight chain alkyl group.

7. Composition according to claim 1, wherein R is 2-ethylhexyl.

8. Composition according to claim 1, wherein the composition is an oral composition and comprises an orally acceptable carrier.

9. Composition according to claim 1, wherein the composition is selected from the group consisting of pastes, gels, foams, liquids, powders and chewing gums and is suitable for use in dental care.

10. Composition according to claim 1, wherein the composition comprises from 0.2 to 10% by weight of the alkyl hydroxybenzoate.

11. Composition according to claim 1, wherein the surfactant is an anionic surfactant.

12. Composition according to claim 1, wherein the surfactant is an alkali-metal alkyl sulphate.

13. Composition according to claim 1, wherein the surfactant is sodium lauryl sulphate.

14. Composition according to claim 1, wherein the surfactant comprises from 1.4 to 1.6% by weight of the composition.
